(19) United States
(12) Patent Application Publication
(10) Pub. No.: US 2004/0058333 A1
(43) Pub. Date: Mar. 25, 2004

(54) Assay products and procedures

(76) Inventor: Michael L. Bell, Fullerton, CA (US)

Correspondence Address: Jefferey G. Sheldon, Sheldon & Mak, 225 South Lake Avenue, 9th floor, Pasadena, CA, 91101, US

(21) Appl. No.: 10/252,474

(22) Filed: Sep. 23, 2002

(51) Int. Cl. C12Q 1/68; G01N 33/53

(52) U.S. Cl. 435/6; 435/7.1

(57) ABSTRACT

In cytometry and like procedures, the number of different types of identifiable particle (and, therefore, the number of detectable analytes) is increased by using the coding labels on some particles as signal labels on other particles, and by using the signal labels on some particles as coding labels on other particles. In one such procedure, the particles of a first class are coded with preselected amounts of a first pair of fluorochromes, and the particles of a second class of particles are coded with preselected amounts of a second pair of fluorochromes. The analytes which interact with the first class of particles are identified by a signal label selected from the second pair; and the analytes which interact with a second class of particles are identified by a signal label selected from the first pair.

### BACKGROUND OF THE INVENTION

1. Field of the Invention

This invention relates to flow cytometry and similar assay procedures.

2. Introduction

In flow cytometry and similar assay procedures, the sample to be assayed is contacted with a multitude of particles. All the particles are coded for recognition purposes and contain analyte-interaction sites which interact selectively with one or more of the analytes in the sample. The particles and the sample are contacted for a time and under conditions such that the desired interaction takes place. The particles fall into different categories. In each category, all the particles

(a) have same coding characteristic, and

(b) contain the same analyte-interaction sites.

The coding characteristic and the analyte-interaction sites in each category are different from those in all the other categories.

Before, during or after the contacting of the particles and the sample, the analytes and/or the sites which have interacted with the analytes are labeled with a signal label. In some assays, an alternative to labeling the analytes and/or the analyte-interaction sites which have interacted with analytes is so-called competitive assay. In a competitive assay, before, during or after the sample has been contacted with the reagent, analogs of the analytes are added to the sample, or to the reagent, or to the mixture of the sample and the reagent; and before, during or after such addition, the analogs and/or the analyte-interaction sites which have interacted with the analogs, are labeled with signal labels. In this specification, when reference is made to a signal label “associated” with an analyte or with an analyte-interaction site which has interacted with an analyte, the signal label can be one which identifies, in any way, interaction between an analyte and an analyte-interaction site, for example a signal label which is

(a) attached to an analyte or to an analyte-interaction site which has interacted with an analyte, or

(b) attached to an analyte analog, or to an analyte-interaction site which has interacted with an analyte analog, as part of a competitive assay.

The particles are then examined with the aid of a cytometer (or like instrument) which recognizes the coding characteristic and the signal labels. In a cytometer, a representative number of particles are randomly and individually examined. On each particle, the coding characteristic identifies the analyte-interaction site, and the signal label(s) identifies (and usually quantifies) the analyte(s). It is also possible to separate the different categories of particle, and then to examine the categories separately.

The coding characteristic on the particles is often provided by securing one or both of two different coding labels to each particle, with the absolute and/or relative amounts of the labels identifying different categories of particle. The cytometer (or other instrument) identifies the relative and/or absolute amounts of the or each coding label. For example, when the coding labels are fluorochromes (which may also be referred to as coding dyes), they are exposed to a laser and produce fluorescence over different wavelength ranges (though the ranges may overlap). The number of different categories of particle which can be assayed depends on (i) the resolving power of the cytometer for the fluorescence (or other characteristic) associated with the coding labels, and (ii) how accurately the desired concentration(s) of the coding labels can be maintained during manufacture and storage of the particles. Different categories of particles can alternatively or additionally be distinguished by other coding characteristics, for example by size, density, radioactivity, color, electrical charge, or magnetic properties.

The particles can be of one or more of three different types, which are referred to herein as single assay particles, associated assay particles, and multi-assay particles.

Single assay particles contain analyte-interaction sites which can interact with (a) only one analyte, or (b) two or more analytes which do not need to be separately assayed and which can, therefore, be associated with the same signal label.

Associated assay particles are a particular class of single assay particles containing analyte-interaction sites which can interact with all the analytes in a group of two or more analytes. The associated assay particles belong to two or more different categories, the number of categories being equal to the number of analytes in the group. The associated assay particles in each category contain analyte-interaction sites which are different from the analyte-interaction sites in each of the other categories. However, all the different analyte-interaction sites can interact with each analyte in the group of analytes, and the affinity of each of the analytes in the group for each of the analyte-interaction sites is known. Under these circumstances, the analytes in the group do not need to be separately assayed on a particle by particle basis, and all the analytes in the group can be associated with the same signal label. This is because the result of examining the associated assay particles in the different categories can be analyzed together to assay each analyte. In essence, the analysis involves the solution of multiple simultaneous equations, each equation resulting from examination of one category of the associated assay particles.

Multi-assay particles contain analyte-interaction sites which interact with two or more analytes which must be separately assayed on each particle. Such particles require different signal labels to be associated with each of the different analytes. Multi-assay particles are usually dual assay particles, i.e. they interact with only two different analytes. One important use of dual assay particles is to assay two different types of rubella antibody, which may for example be labeled by different signal dyes before, during or after they interact with the particles.

Flow cytometers are often designed so that they can carry out both assays in which only single assay particles are used and assays in which both single assay and dual assay particles are used. One type of conventional flow cytometer comprises

1) a first laser (often called a coding laser) which causes fluorescence of first and second coding dyes;

2) a first fluorescence assessment system which has a first detection channel for assessing fluorescence produced by the first coding dye and a second detection channel for assessing fluorescence produced by the second coding dye;

3) a second laser (often called a detection laser) which causes fluorescence of signal dye(s) associated with the analyte(s); and

4) a second fluorescence assessment system which has

(i) a first detection channel for assessing fluorescence associated with the signal dye which is associated with the single assay particles and which is one of two signal dyes associated with the dual assay particles, and

(ii) a second detection channel for assessing fluorescence associated with the other signal dye associated with dual assay particles.

Another type of conventional flow cytometer comprises

1) a laser which causes fluorescence of the coding dyes and the signal dyes associated with the analytes

2) a first fluorescence assessment system which has a first detection channel for assessing the fluorescence produced by a first coding dye and a second detection channel for assessing the fluorescence produced by a second coding dye; and

3) a second fluorescence assessment system which has a first detection channel for assessing the fluorescence from the signal dye which is associated with single assay particles and which is one of the two signal dyes on dual assay particles, and a second detection channel for assessing the fluorescence from the other signal dyes associated with dual assay particles.

When the reagent composition contains only single assay particles, only three of the four detection channels in these conventional cytometers are used. When the reagent composition contains both single assay and multi-assay particles, only three of the four detection channels are used for much of the time.

For disclosure of cytometry and similar assay procedures, reference may be made for example to U.S. Pat. No. 4,499,052 (Fulwyler), U.S. Pat. No. 4,665,020 (Saunders), U.S. Pat. No. 4,699,828 (Schwartz et al), U.S. Pat. No. 5,028,545 (Soini), U.S. Pat. No. 5,073,497 (Schwartz), U.S. Pat. No. 5,747,349 (van den Engh et al), U.S. Pat. No. 5,981,180 (Chandler et al.), U.S. Pat. No. 6,023,540 (Walt et al), U.S. Pat. No. 6,159,748 (Hechinger) and U.S. Pat. No. 6,165,796 (Bell), European Patent No. 126, 450, WO 01/13120, and copending, commonly assigned, U.S. application Ser. No. 09/991,001, filed Nov. 14, 2001, the entire disclosures of which are incorporated by reference herein for all purposes.

### SUMMARY OF THE INVENTION

I have realized, in accordance with the present invention, that by varying the functions of the labels conventionally used as coding and signal labels in flow cytometry and like procedures,

a) the number of different categories of particle (and, therefore, the number of analytes) can be increased, and/or

b) the resolution required in the detection channels can be decreased, and/or

c) the permissible variability of the particles (in production and/or after storage) can be increased.

Furthermore, the present invention can be used to increase the capabilities of conventional cytometers without any change in their physical construction.

In a first aspect, this invention provides a composition which is suitable for use as a reagent in assaying a sample, the composition comprising a plurality of particles, each of the particles

(i) having a coding characteristic,

(ii) containing analyte-interaction sites, and

(iii) belonging to one only of a plurality of defined categories, and each of the particles in each defined category

(a) having the same coding characteristic, the coding characteristic being provided by a single coding label in a preselected amount or by two or more coding labels in preselected amounts, the coding label or labels being selected from a group of at least n labels, where n is at least 3, all of the labels being capable of being assessed in the same way, the number of coding labels on each particle being at most (n−1), and each of the labels in the group being a coding label in at least one of the defined categories, and

(b) containing the same analyte-interaction sites;

the combination of the coding characteristic and the analyte-interaction sites on the particles in each category being different from the combination of the coding characteristic and the analyte-interaction sites in all the other categories.

In a second aspect, this invention provides a composition which is a precursor for a composition according to the first aspect of the invention, and which is the same as a composition according to the first aspect of the invention except that at least some of the particles contain, in place of the analyte-interaction sites, precursors for analyte-interaction sites. The compositions of the second aspect of the invention can be converted into compositions of the first aspect of the invention by one or more appropriate steps.

In a third aspect, this invention provides a composition which can be examined in a cytometer or like instrument, the composition comprising a plurality of particles, each of the particles

(i) having a coding characteristic,

(ii) containing analyte-interaction sites and/or corresponding sites which are the same as said analyte-interaction sites except that they have interacted with one or more analytes, and

(iii) belonging to one only of a plurality of defined categories, each of the particles in each defined category

(a) having the same coding characteristic, the coding characteristic being provided by a single coding label in a preselected amount or by two or more coding labels in preselected amounts, the coding label or labels being selected from a group of at least n labels, where n is at least 3, all of the labels being capable of being assessed in the same way, the number of coding labels on each particle being at most (n−1), and each of the labels in the group being a coding label in at least one of the defined categories, and

(b) containing the same analyte-interaction sites and/or corresponding sites which have interacted with one or more analytes;

the combination of the coding characteristic and the analyte-interaction sites and/or corresponding sites on the particles in each category being different from combination of the coding characteristic and the analyte-interaction sites and/or corresponding sites on the particles in all the other categories; and

the particles in at least one of the categories containing analyte-interaction sites which have interacted with one or more analytes and which are associated with a signal label which (i) is selected from the group of at least n labels and (ii) is not one of the labels which provides the coding characteristic for the particle containing the interacted site.

In a fourth aspect, this invention provides a method of analyzing a sample to ascertain whether it contains possible analytes, the method comprising

(A) contacting the sample with a composition according to the first aspect of the invention;

(B) carrying out a treatment which results in the association of a signal label with the sites which have interacted with one or more analytes, the signal label (i) being selected from the group of at least n labels and (ii) not being one of the labels which provides the coding characteristic for the particles carrying the interacted sites; and

(C) after steps (A) and (B), identifying the coding and signal labels on each particle of a representative sample of the particles.

In a fifth aspect, this invention provides a cytometer or like instrument comprising a computer which is programmed so that the instrument will examine a composition according to the third aspect of the invention and produce an assay of the analytes which have interacted with the analyte-interaction sites.

In a sixth aspect, this invention provides software which can be installed on a computer controlling a cytometer or like instrument so that the instrument will examine a composition according to the third aspect of the invention and produce an assay of the analytes which have interacted with the analyte-interaction sites.

### DETAILED DESCRIPTION OF THE INVENTION

In the Summary of the Invention above and in the Detailed Description of the Invention below, reference is made to particular features (including method steps) of the invention. It is to be understood that the disclosure of the invention in this specification includes all appropriate combinations of such particular features. For example, where a particular feature is disclosed in the context of a particular aspect or embodiment of the invention, that feature can also be used, to the extent appropriate, in combination with and/or in the context of other particular aspects and embodiments of the invention, and in the invention generally.

In describing and claiming the invention below, the following definitions (in addition to those already given) are used. The term “comprises” (and grammatical variations thereof) in relation to methods, materials, things etc is used herein to mean that the methods, materials, things etc. can optionally include, in addition to the steps, features, components, etc. explicitly specified after the term “comprises” (and grammatical variations thereof), other steps, features, ingredients, etc. Where reference is made herein to a method comprising two or more steps, the steps can be carried out in any order, or simultaneously, except where the context excludes that possibility. The term “at least” followed by a number is used herein to denote the start of a range beginning with that number (which may be a range having an upper limit or no upper limit, depending on the variable being defined). For example “at least 3” means 3 or more than 3. The term “at most” followed by a number is used herein to denote the end of a range ending with that number (which may be a range having 1 or 0 as its lower limit, or a range having no lower limit, depending upon the variable being defined). For example, “at most 4” means 4 or less than 4. When, in this specification, a range is given as “(a first number) to (a second number)” or “(a first number)—(a second number)”, this means a range whose lower limit is the first number and whose upper limit is the second number.

The term “analyte-interaction sites” is used herein to denote a site which will interact with one or more selected analytes in a way in which (i) results in the association of a signal label with the analyte-interaction site or (ii) which makes it possible for later steps to cause a signal label to be associated with the analyte-interaction site. The interaction can be of any kind, for example formation of a covalent, coordinate or ionic bond, hybridisation of nucleotides, or enzymatic action. The change produced by the interaction can for example results in the creation or modification of fluorescence or another property which can be examined in a cytometer or a similar instrument.

The coding and signal labels used in the present invention must be such that they can be assessed in the same way. Examples of such labels are those that can be assessed through their fluorescence, chemiluminescence, or absorption characteristics. Many of the labels currently available are fluorochromes, which are assessed through their fluorescence when exposed to a laser. The fluorochromes presently used as coding dyes often form a pair, in that both fluoresce when exposed to the same laser and often fluoresce in different but adjacent or overlapping wavelength bands. Similarly, the fluorochromes presently used as signal dyes often form a pair, in that both fluoresce when exposed to the same laser (which may be the same as or different from the laser used to cause the coding dyes to fluoresce) and often fluoresce in different but adjacent or overlapping wavelength bands which are different from the wavelength bands in which the coding dyes fluoresce.

The total number of labels used to provide the coding and signal labels in this invention (the number n in the definition above of the first aspect of the invention) is at least 3. The value of n is less than or equal to the number of detection channels available in the cytometer, for example from 3 to 6. Preferably, the value of n is equal to the number of detection channels, since this maximizes the number of identifiable particles. Many conventional cytometers, for example cytometers of the two types described above, have four detection channels.

As noted above, the coding characteristic of the particles can be provided by a single coding label in a preselected amount or by two or more coding labels in preselected amounts. This includes the possibility that the coding characteristic is a function of the absolute and/or relative amounts of the coding label(s). For example, particles in two different categories can be coded by (i) two different amounts of a single coding label or (ii) two coding labels which are present in the same ratio in both categories, but in amounts which are different in the two categories.

In the various aspects of the invention, as defined above, the labels can be allocated to the coding and signal functions in a wide variety of combinations, with the number of possible combinations increasing rapidly as the value of n increases. Some of these combinations may cause misleading results, because it will not be possible to determine whether a particular reading results from a coding label which is present in a preselected amount or a signal label which is present in a variable amount determined by the concentration of the relevant analyte. Those skilled in the art will have no difficulty, having regard to the disclosure in this specification and their own knowledge, in ascertaining the groups (including pairs) of particles that may give rise to false results. The possibility of false results can be eliminated by using only one (or none) of the particles in the group, or by placing additional coding on the particles. Generally, it is preferred that each particle is coded by one or two coding labels, particularly two coding labels.

In one embodiment, the number of labels in the group (n) is four. Two of the labels may form a first pair, for example a pair of labels conventionally used as coding labels, and the other two of the labels may form a second pair, for example a pair of labels conventionally used as signal labels. When using such pairs of labels, preferably all the particles are coded with one or both (particularly both) of the labels of the first pair or with one or both (particularly both) of the labels of the second pair. However, the invention also includes coding some of the particles with one of the first pair of labels and one of the second pair of labels.

In this embodiment, if the coding of the particles is provided only by the coding labels, the compositions of the first aspect of the invention preferably comprise a plurality of particles, each of the particles containing analyte-interaction sites and belonging to a first class (AB) or to a second class (XY),

each of the particles of class AB belonging to one of two or more subclasses (AB₁, AB₂ . . . ABₙ),

each of the particles in each subclass AB₁, AB₂ . . . ABₙ being coded by at least one of a first label A and a second label B, the amounts of the labels A and B being such that the particles of each sub class can be distinguished from the particles of the other subclasses AB₁, AB₂ . . . ABₙ, and

each of the particles in each subclass AB₁, AB₂ . . . ABₙ belonging to one of two subsubclasses (AB_{1X}, AB_{1Y}), (AB_{2X}, AB_{2Y}) . . . (AB_{nX}, AB_{nY}), and

each of the particles of class XY belonging to one of two or more subclasses (XY₁, XY₂ . . . XYₙ),

each of the particles in each subclass XY₁, XY₂ . . . XYₙ being coded by at least one of a third label X and a fourth label Y, the amounts of the labels X and Y being such that the particles in each sub class can be distinguished from the particles of the other subclasses XY₁, XY₂ . . . XYₙ; and

each of the particles in each subclass XY₁, XY₂ . . . XYₙ belonging to one of two subsubclasses (XY_{1A}, XY_{1B}), (XY_{2A}, XY_{2B}) . . . (XY_{nA}, XY_{nB}),

the analyte-interaction sites contained by the particles in each of the subsubclasses (AB₁ₓ, AB_{1Y}), (AB_{2X}, AB_{2Y}) . . . (AB_{nX}, AB_{nY}) and (XY_{1A}, XY_{1B}), (XY_{2A}, XY_{2B}) . . . (XY_{nA}, XY_{nB}), being different from the analyte-interaction sites contained by the particles in the other subsubclasses.

Correspondingly, in this embodiment, the compositions of the third aspect of the invention are the same as the compositions of the first aspect of the invention except that analyte-interaction sites on the particles in at least one of the subsubclasses have interacted with one or more analytes, and those interacted sites are associated with a signal label which is

(i) for the analyte-interaction sites on the particles of class AB, the third label X for one of the subsubclasses of each subclass and the fourth label Y for the other of the subsubclasses of that subclass, and

(ii) for the analyte-interaction sites on the particles of class XY, the first label A for one of the subsubclasses of each subclass and the second label B for the other of the subsubclasses of each subclass;

Correspondingly, in this embodiment, the method of the fourth aspect of the invention comprises p1 (A) contacting the sample to be assayed with a composition of the first aspect of the invention as defined above for this embodiment;

(B) before, during or after steps (A), carrying out a treatment which results in the association of a signal label with the analyte-interaction sites which have interacted with one or more analytes, the signal label being

(i) for the analyte-interaction sites on the particles of class AB, the third label X for one of the subsubclasses of each subclass and the fourth label Y for the other of the subsubclasses of that subclass, and

(ii) for the analyte-interaction sites on the particles of class XY, the first label A for one of the subsubclasses of each subclass and the second label B for the other of the subsubclasses of each subclass; and

(C) after steps (A) and (B), identifying the coding and signal labels on each particle of a representative sample of the particles.

Step B of this method can for example comprise

(B1) before, during or after step (A), carrying out a treatment which, for each of the possible analytes and group or groups of analytes, if present, results in the presence of a signal label as defined on analyte-interaction sites which have interacted with the analyte or one of the group or groups of analytes; or

(B2) before, during or after step (A), contacting the sample, or the reagent, or the product of step (A), with a composition comprising an analog for each of the possible analytes or group of possible analytes, whereby analyte-interaction sites which have not interacted with the analytes interact with said analogs, and before, during or after said contacting, carrying out a treatment which, for each analog, results in the presence of a signal label as defined on the analyte-interaction sites which have interacted with one of the analogs.

The Tables below will assist in understanding the invention. The Tables set out, by way of diagrammatic example, fluorescence values that could be recorded in the four channels of a cytometer when using various different particles, coding dyes, analytes, and signal dyes. In practice, a pair of coding dyes may be used in absolute and relative amounts such that many more different classes of particles can be distinguished from each other, for example up to 100 classes or even more. However, in the Tables, for the sake of brevity and simplicity, the coding dyes are used in only four different ratios (0:100, 33:67, 67:33 and 100:0). The conclusions which can be drawn from the Tables below are the same, in principle, as they would be if the coding dyes were used in absolute and relative amounts such that many more different classes of particles were used. The numerical fluorescence values given in the Tables correspond to the proportions of the coding dyes in the particles (though in practice, the relative strengths of the fluorescences may not correspond to the relative amounts of the fluorochromes), and the abbreviation VAR corresponds to the unknown concentration of the analyte which interacts with the particle.

Tables P1 and P2 represent prior art assays. In Tables P1 and P2, the particles are (i) coded with one or both of two different dyes (A and B) and (ii) contain analyte-interaction sites which interact with different analytes (An1P, An1Q, An2, An3 and An4 in Table P1, An1, An2, An3 and An4 in Table P2). Each analyte is identified (before, during or after interaction) by a signal dye (X and Y in Table A, X in Table B). Tables 1-7 represent assays in accordance with the invention. In Tables 1-7, the particles (i) are coded with one or two of a first pair of dyes (A and B) or with one or two of a second pair of dyes (X and Y) in different ratios, and (ii) contain interaction sites which interact with different analytes (An1, An2 . . . An16). Each analyte is identified (before or after interaction) by one of the four dyes A, B, X and Y, selected in accordance with the invention, i.e. one of X and Y is the signal dye for the particles coded with one or both of A and B, and one of A and B is the signal dye for the particles coded with one or both of X and Y. There are eight types of particle in class AB, which contains four subclasses, each having two subsubclasses; similarly, there are eight types of particle in class XY, which contains four subclasses, each having two subsubclasses.

In Table 1, all the particles are single assay particles, and for each of the subsubclasses, there are two possibilities, namely that the analyte with which the subsubclass will interact is present or absent, resulting in the thirty-two “possible particles” referred to in the table. As shown by Table 1, the fact that some of the particles are coded with only one of the coding dyes can give rise to the possibility of false results. For example, “possible particles” 2 and 32 give rise to fluorescence in channels B and X of the “100 and VAR” and “VAR and 100” respectively. If the values of VAR are such that it is impossible to distinguish between these two results, and there is no other way to distinguish the particles, then false results will be obtained. The same is true for “possible particles” 8 and 24, 10 and 26, and 16 and 18 respectively. The possibility of such false results can be eliminated by using only one of the particles that can give rise to confusing results. For example, if the particles coded with only one of coding dyes X and Y are not used, then the possibility of false results is eliminated, but only 12 analytes can be assayed. This is illustrated in Table 2. Similarly, if the particles coded with only one of coding dyes A and B are not used, the possibility of false results is eliminated. More simply (but further reducing the number of analytes that can be assayed to 8) the possibility of false results can be eliminated by using only particles which are coded with two coding dyes. This is illustrated in Table 3.

Table 4 is the same as Table 1, except that the particles in subsubclass AB_{1X} (coded with only one coding dye) are dual assay particles which react with analytes An1P and An1Q. As will be seen from Table 4, the inclusion of dual assay particles increases the possibility of false results. Thus, the “possible particles” 2(i) and 32; 2(ii), 10 and 26; 2(iii), 28 and 30; 8 and 24; and 16 and 18, respectively can be confused with each other, unless it is possible to distinguish between the values 100 and VAR (or otherwise to distinguish between the particles in question). Again, the possibility of false results can be eliminated by using only one of the particles which can be confused. If all the particles except the dual assay particle are coded with two coding dyes, the possibility of confusing results is reduced but not completely eliminated.

As is shown by a comparison of Table 4 and Tables 5-7 below, if some of the particles are dual assay particles, it is preferable that the dual assay particles should be coded with two coding labels, rather than with a single coding label, since coding of dual assay particles with two labels reduces the number of coding combinations which can give rise to confusing results.

Table 5 is similar to Table 4, in that it includes a subsubclass of dual assay particles. However, in Table 5 the dual assay particles are coded with two coding dyes. Table 5 is also the same as Table 1, except that the particles in subsubclass AB_{2X} (coded with two coding dyes) are dual assay particles which react with analytes An2P and An2Q. As will be seen from Table 5, the possibility of false results is greater than in Table 1, but less than in Table 4. Thus, in Table 5, not only can “possible particles” 2 and 32, 8 and 24, 10 and 26, and 16 and 18 respectively, be confused with each other (as in Table 1), but so also can “possible particles” 4 (i) and 12 be confused (unless it is possible to distinguish between the values 100 and VAR, or otherwise to distinguish between the particles in question). Again, the possibility of false results can be eliminated by using only one of the particles which can give rise to confusing results. For example, if the particles coded with only one of coding dyes X and Y are not used, and the particles having the same coding dyes in the same ratio as the dual assay particles are not used, then the possibility of false results is eliminated. However, only 12 analytes can be assayed. This is illustrated in Table 6. Similarly, if the particles coded with only one of coding dyes A and B are not used, the possibility of false results is eliminated. More simply (but further reducing the number of analytes that can be assayed to 8), the possibility of false results can be eliminated by using only particles which are coded with two coding dyes. This is illustrated in Table 7.

It is pointed out that, as noted above, it is often possible to use a pair of coding dyes in many more distinguishable ratios than are shown in the Tables below. In such circumstances, elimination of particles coded with only one of one of the pairs of coding dyes (as in Tables 2 and 6) or elimination of particles coded with only one coding dye (as in Tables 3 and 7) results in a smaller proportionate reduction in the number of analytes which can be assayed.

TABLE P1
(Prior Art)
All particles are coded with one or both of 2 coding dyes, and one of
the particles coded with only one coding dye is a dual assay particle

| Particle | Particle coding dye | | | | Inter- | An | Possible | Fluorescence in Channel | | | |
|---|---|---|---|---|---|---|---|---|---|---|---|
| Type | A | B | X | Y | acts + | signal | particles | A | B | X | Y |
| 1 | 0 | 100 | 0 | 0 | An1P & | X | 1. — | 0 | 100 | 0 | 0 |
| | | | | | An1Q | Y | 2i An1P | 0 | 100 | VAR | 0 |
| | | | | | | | 2ii An1Q | 0 | 100 | 0 | VAR |
| | | | | | | | 2iii An1P & | 0 | 100 | VAR | VAR |
| | | | | | | | An1Q | | | | |
| 2 | 33 | 67 | 0 | 0 | An2 | X | 3. — | 33 | 67 | 0 | 0 |
| | | | | | | | 4. An 2 | 33 | 67 | VAR | 0 |
| 3 | 67 | 33 | 0 | 0 | An3 | X | 5. — | 67 | 33 | 0 | 0 |
| | | | | | | | 6. An3 | 67 | 33 | VAR | 0 |
| 4 | 100 | 0 | 0 | 0 | An4 | X | 7. — | 100 | 0 | 0 | 0 |
| | | | | | | | 8. An4 | 100 | 0 | VAR | 0 |

TABLE P2
(Prior Art)
All particles are single assay particles coded
with one or both of 2 coding dyes.

| Particle | Particle coding dye | | | | Inter- | An | Possible | Fluorescence in Channel | | | |
|---|---|---|---|---|---|---|---|---|---|---|---|
| Type | A | B | X | Y | acts + | signal | particles | A | B | X | Y |
| 1 | 0 | 100 | 0 | 0 | An1 | X | 1. — | 0 | 100 | 0 | 0 |
| | | | | | | | 2 An1 | 0 | 100 | VAR | 0 |
| 2 | 33 | 67 | 0 | 0 | An2 | X | 3. — | 33 | 67 | 0 | 0 |
| | | | | | | | 4. An 2 | 33 | 67 | VAR | 0 |
| 3 | 67 | 33 | 0 | 0 | An3 | X | 5. — | 67 | 33 | 0 | 0 |
| | | | | | | | 6. An3 | 67 | 33 | VAR | 0 |
| 4 | 100 | 0 | 0 | 0 | An4 | X | 7. — | 100 | 0 | 0 | 0 |
| | | | | | | | 8. An4 | 100 | 0 | VAR | 0 |

TABLE 1
All particles are single assay particles and are coded with one or both of a pair of
coding dyes AB or with one or both of a pair of coding dyes XY. Each analyte is
identified by a single signal dye selected from the pair of dyes not used as coding
dyes on the particle with which the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{1X} | 0 | 100 | 0 | 0 | An1 | X | 1. — | 0 | 100 | 0 | 0 |
| | | | | | | | 2. An1 | 0 | 100 | VAR | 0 |
| AB_{2X} | 33 | 67 | 0 | 0 | An2 | X | 3. — | 33 | 67 | 0 | 0 |
| | | | | | | | 4. An 2 | 33 | 67 | VAR | 0 |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{4X} | 100 | 0 | 0 | 0 | An4 | X | 7. — | 100 | 0 | 0 | 0 |
| | | | | | | | 8. An4 | 100 | 0 | VAR | 0 |
| AB_{1Y} | 0 | 100 | 0 | 0 | An5 | Y | 9. — | 0 | 100 | 0 | 0 |
| | | | | | | | 10. An5 | 0 | 100 | 0 | VAR |
| AB_{2Y} | 33 | 67 | 0 | 0 | An6 | Y | 11. — | 33 | 67 | 0 | 0 |
| | | | | | | | 12. An6 | 33 | 67 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| AB_{4Y} | 100 | 0 | 0 | 0 | An8 | Y | 15. — | 100 | 0 | 0 | 0 |
| | | | | | | | 16. An8 | 100 | 0 | 0 | VAR |
| XY_{1A} | 0 | 0 | 0 | 100 | An9 | A | 17. — | 0 | 0 | 0 | 100 |
| | | | | | | | 18. An9 | VAR | 0 | 0 | 100 |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{4A} | 0 | 0 | 100 | 0 | An12 | A | 23. — | 0 | 0 | 100 | 0 |
| | | | | | | | 24. An12 | VAR | 0 | 100 | 0 |
| XY_{1B} | 0 | 0 | 0 | 100 | An13 | B | 25. — | 0 | 0 | 0 | 100 |
| | | | | | | | 26. An13 | 0 | VAR | 0 | 100 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |
| XY_{4B} | 0 | 0 | 100 | 0 | An16 | B | 31. — | 0 | 0 | 100 | 0 |
| | | | | | | | 32. An16 | 0 | VAR | 100 | 0 |

TABLE 2
All particles are single assay particles and are coded with one or both of a pair of
coding dyes AB or with both of a pair of coding dyes XY. Each analyte is identi-
fied by a single signal dye selected from the pair of dyes not used as coding dyes
on the particle with which the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{1X} | 0 | 100 | 0 | 0 | An1 | X | 1. — | 0 | 100 | 0 | 0 |
| | | | | | | | 2. An1 | 0 | 100 | VAR | 0 |
| AB_{2X} | 33 | 67 | 0 | 0 | An2 | X | 3. — | 33 | 67 | 0 | 0 |
| | | | | | | | 4. An 2 | 33 | 67 | VAR | 0 |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{4X} | 100 | 0 | 0 | 0 | An4 | X | 7. — | 100 | 0 | 0 | 0 |
| | | | | | | | 8. An4 | 100 | 0 | VAR | 0 |
| AB_{1Y} | 0 | 100 | 0 | 0 | An5 | Y | 9. — | 0 | 100 | 0 | 0 |
| | | | | | | | 10. An5 | 0 | 100 | 0 | VAR |
| AB_{2Y} | 33 | 67 | 0 | 0 | An6 | Y | 11. — | 33 | 67 | 0 | 0 |
| | | | | | | | 12. An6 | 33 | 67 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| AB_{4Y} | 100 | 0 | 0 | 0 | An8 | Y | 15. — | 100 | 0 | 0 | 0 |
| | | | | | | | 16. An8 | 100 | 0 | 0 | VAR |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |

TABLE 3
All particles are single assay particles and are coded with both of a pair of coding
dyes AB or with both of a pair of coding dyes XY. Each analyte is identified by a
single signal dye selected from the pair of dyes not used as coding dyes on the
particle with which the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{2X} | 33 | 67 | 0 | 0 | An2 | X | 3. — | 33 | 67 | 0 | 0 |
| | | | | | | | 4. An 2 | 33 | 67 | VAR | 0 |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{2Y} | 33 | 67 | 0 | 0 | An6 | Y | 11. — | 33 | 67 | 0 | 0 |
| | | | | | | | 12. An6 | 33 | 67 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |

TABLE 4
All particles are coded with one or both of a pair of coding dyes AB or one or with
both of a pair of coding dyes XY. One of the particles coded with only one coding
dye is a dual assay particle. Each analyte is identified by a single signal dye
selected from the pair of dyes not used as coding dyes on the particle with which
the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{1X} | 0 | 100 | 0 | 0 | An1P & | X | 1. — | 0 | 100 | 0 | 0 |
| | | | | | An1Q | Y | 2i An1P | 0 | 100 | VAR | 0 |
| | | | | | | | 2ii An1Q | 0 | 100 | 0 | VAR |
| | | | | | | | 2iii An1P & | 0 | 100 | VAR | VAR |
| | | | | | | | AN1Q | | | | |
| AB_{2X} | 33 | 67 | 0 | 0 | An2 | X | 3. — | 33 | 67 | 0 | 0 |
| | | | | | | | 4. An 2 | 33 | 67 | VAR | 0 |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{4X} | 100 | 0 | 0 | 0 | An4 | X | 7. — | 100 | 0 | 0 | 0 |
| | | | | | | | 8. An4 | 100 | 0 | VAR | 0 |
| AB_{1Y} | 0 | 100 | 0 | 0 | An5 | Y | 9. — | 0 | 100 | 0 | 0 |
| | | | | | | | 10. An5 | 0 | 100 | 0 | VAR |
| AB_{2Y} | 33 | 67 | 0 | 0 | An6 | Y | 11. — | 33 | 67 | 0 | 0 |
| | | | | | | | 12. An6 | 33 | 67 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| AB_{4Y} | 100 | 0 | 0 | 0 | An8 | Y | 15. — | 100 | 0 | 0 | 0 |
| | | | | | | | 16. An8 | 100 | 0 | 0 | VAR |
| XY_{1A} | 0 | 0 | 0 | 100 | An9 | A | 17. — | 0 | 0 | 0 | 100 |
| | | | | | | | 18. An9 | VAR | 0 | 0 | 100 |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{4A} | 0 | 0 | 100 | 0 | An12 | A | 23. — | 0 | 0 | 100 | 0 |
| | | | | | | | 24. An12 | VAR | 0 | 100 | 0 |
| XY_{1B} | 0 | 0 | 0 | 100 | An13 | B | 25. — | 0 | 0 | 0 | 100 |
| | | | | | | | 26. An13 | 0 | VAR | 0 | 100 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |
| XY_{4B} | 0 | 0 | 100 | 0 | An16 | B | 31. — | 0 | 0 | 100 | 0 |
| | | | | | | | 32. An16 | 0 | VAR | 100 | 0 |

TABLE 5
All particles are coded with one or both of a pair of coding dyes AB or with one
or both of a pair of coding dyes XY. One of the particles coded with two coding
dyes is a dual assay particle. Each analyte is identified by a single signal dye
selected from the pair of dyes not used as coding dyes on the particle with which
the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{1X} | 0 | 100 | 0 | 0 | An1 | X | 1. — | 0 | 100 | 0 | 0 |
| | | | | | | | 2. An1 | 0 | 100 | VAR | 0 |
| AB_{2X} | 33 | 67 | 0 | 0 | An2P | X | 3. — | 33 | 67 | 0 | 0 |
| | | | | | An2Q | Y | 4i An 2P | 33 | 67 | VAR | 0 |
| | | | | | | | 4ii An2Q | 33 | 67 | 0 | VAR |
| | | | | | | | 4iii An2 & | 33 | 67 | VAR | VAR |
| | | | | | | | An2Q | | | | |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{4X} | 100 | 0 | 0 | 0 | An4 | X | 7. — | 100 | 0 | 0 | 0 |
| | | | | | | | 8. An4 | 100 | 0 | VAR | 0 |
| AB_{1Y} | 0 | 100 | 0 | 0 | An5 | Y | 9. — | 0 | 100 | 0 | 0 |
| | | | | | | | 10. An5 | 0 | 100 | 0 | VAR |
| AB_{2Y} | 33 | 67 | 0 | 0 | An6 | Y | 11. — | 33 | 67 | 0 | 0 |
| | | | | | | | 12. An6 | 33 | 67 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| AB_{4Y} | 100 | 0 | 0 | 0 | An8 | Y | 15. — | 100 | 0 | 0 | 0 |
| | | | | | | | 16. An8 | 100 | 0 | 0 | VAR |
| XY_{1A} | 0 | 0 | 0 | 100 | An9 | A | 17. — | 0 | 0 | 0 | 100 |
| | | | | | | | 18. An9 | VAR | 0 | 0 | 100 |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{4A} | 0 | 0 | 100 | 0 | An12 | A | 23. — | 0 | 0 | 100 | 0 |
| | | | | | | | 24. An12 | VAR | 0 | 100 | 0 |
| XY_{1B} | 0 | 0 | 0 | 100 | An13 | B | 25. — | 0 | 0 | 0 | 100 |
| | | | | | | | 26. An13 | 0 | VAR | 0 | 100 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |
| XY_{4B} | 0 | 0 | 100 | 0 | An16 | B | 31. — | 0 | 0 | 100 | 0 |
| | | | | | | | 32. An16 | 0 | VAR | 100 | 0 |

TABLE 6
All particles are coded with one or both of a pair of coding dyes AB or with both
of a pair of coding dyes XY. One of the particles is coded with two coding dyes and
is a dual assay particle. No other particles have the same coding dyes in the same
ratio as the dual assay particle. Each analyte is identified by a single signal dye
selected from the pair of dyes not used as coding dyes on the particle with which the
analyte interacts. One member of each pair of particles giving rise to the possibility
of confusion has been eliminated.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{1X} | 0 | 100 | 0 | 0 | An1 | X | 1. — | 0 | 100 | 0 | 0 |
| | | | | | | | 2. An1 | 0 | 100 | VAR | 0 |
| AB_{2X} | 33 | 67 | 0 | 0 | An2P | X | 3. — | 33 | 67 | 0 | 0 |
| | | | | | An2Q | Y | 4i An 2P | 33 | 67 | VAR | 0 |
| | | | | | | | 4ii An2Q | 33 | 67 | 0 | VAR |
| | | | | | | | 4iii An2 & | 33 | 67 | VAR | VAR |
| | | | | | | | An2Q | | | | |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{4X} | 100 | 0 | 0 | 0 | An4 | X | 7. — | 100 | 0 | 0 | 0 |
| | | | | | | | 8. An4 | 100 | 0 | VAR | 0 |
| AB_{1Y} | 0 | 100 | 0 | 0 | An5 | Y | 9. — | 0 | 100 | 0 | 0 |
| | | | | | | | 10. An5 | 0 | 100 | 0 | VAR |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| AB_{4Y} | 100 | 0 | 0 | 0 | An8 | Y | 15. — | 100 | 0 | 0 | 0 |
| | | | | | | | 16. An8 | 100 | 0 | 0 | VAR |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{2B} | 0 | 0 | 33 | 67 | An14 | B | 23. — | 0 | 0 | 33 | 67 |
| | | | | | | | 24. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |

TABLE 7
All particles are coded with both of a pair of coding dyes AB or with both of a pair
of coding dyes XY. One of the particles is a dual assay particle. No other particles
have the same coding dyes in the same ratio as the dual assay particle. Each analyte
is identified by a single signal dye selected from the pair of dyes not used as coding
dyes on the particle with which the analyte interacts.

| Particle | | | | | | | | | | | |
|---|---|---|---|---|---|---|---|---|---|---|---|
| subsub | Particle coding dye | | | | Inter- | Analyte | Possible | Fluorescence in Channel | | | |
| class | A | B | X | Y | acts + | signal | particles | A | B | X | Y |
| AB_{2X} | 33 | 67 | 0 | 0 | An2P | X | 3. — | 33 | 67 | 0 | 0 |
| | | | | | An2Q | Y | 4i. An 2 | 33 | 67 | VAR | 0 |
| | | | | | | | 4ii. An2Q | 33 | 67 | 0 | VAR |
| | | | | | | | 4iii. An2 & | 33 | 67 | VAR | VAR |
| | | | | | | | An2Q | | | | |
| AB_{3X} | 67 | 33 | 0 | 0 | An3 | X | 5. — | 67 | 33 | 0 | 0 |
| | | | | | | | 6. An3 | 67 | 33 | VAR | 0 |
| AB_{3Y} | 67 | 33 | 0 | 0 | An7 | Y | 13. — | 67 | 33 | 0 | 0 |
| | | | | | | | 14. An7 | 67 | 33 | 0 | VAR |
| XY_{2A} | 0 | 0 | 33 | 67 | An10 | A | 19. — | 0 | 0 | 33 | 67 |
| | | | | | | | 20. An10 | VAR | 0 | 33 | 67 |
| XY_{3A} | 0 | 0 | 67 | 33 | An11 | A | 21. — | 0 | 0 | 67 | 33 |
| | | | | | | | 22. An11 | VAR | 0 | 67 | 33 |
| XY_{2B} | 0 | 0 | 33 | 67 | An14 | B | 27. — | 0 | 0 | 33 | 67 |
| | | | | | | | 28. An14 | 0 | VAR | 33 | 67 |
| XY_{3B} | 0 | 0 | 67 | 33 | An15 | B | 29. — | 0 | 0 | 67 | 33 |
| | | | | | | | 30. An15 | 0 | VAR | 67 | 33 |

Table 7 is a particular example of the results obtained in an embodiment of the invention in which

1) the labels are provided by a first pair of fluorochromes, AB, and a second pair of fluorochromes, XY;

2) all the particles in the reagent composition are

((i) single assay particles coded by both labels of the first pair or by both labels of the second pair, or

(ii) dual assay particles coded by both labels of the first pair.

The reagent composition is mixed with a sample which may contain analytes which will interact with the respective analyte-interaction sites on the different categories of particle. If the mixing of the reagent and the sample does not cause the analyte-interaction sites which have interacted with analytes to be labeled with signal labels, the mixture is treated to cause signal labels to be associated with the analyte-interaction sites which have interacted with analytes. The signal label associated with the single assay particles is one of the pair of labels not used to code the particle. The two signal labels associated respectively with the two different analytes which have interacted with the analyte-interaction sites on the dual assay particles are the two labels of the second pair.

The resulting labeled particles are then processed in a four-channel cytometer, for example a cytometer having a first laser which causes the first pair of labels to fluoresce and a second laser which causes the second pair of labels to fluoresce. For each particle, the reading process first involves determining whether the level of fluorescence in each channel is high enough to be significant. This can be done in a variety of ways. A simple way is to set a threshold value and to say that the fluorescence is significant if it exceeds that value. If the A, B, X and Y channels are significant, this shows that the particle is a dual assay particle and that the sample contains both the analytes which interact with the analyte-interaction sites on the dual assay particle. The AB channels determine the particle category and the X and Y channels determine the concentrations of the two analytes. If only the A, B and X or A, B and Y channels are significant; the particle may be a dual assay particle or a single assay particle; the AB channels identify the particle category; for the single assay particles, the sample contains the analyte which interacts with the analyte-interaction sites on the identified particles, and the X or Y channel determines the concentration of that analyte; and for the dual assay particles, the sample contains one of the two analytes which interact with the analyte-interaction sites on the identified particle, and the X or Y channel identifies which of the two analytes has interacted and determines its concentration. If only the A, X and Y or B, X and Y channels are significant; the particle is a single assay particle; the XY channels identify the particle category; the sample contains the analyte which interacts with the analyte-interaction sites on the identified particles, and the A or B channel determines the concentration of that analyte. If there are two or fewer significant channels, the reading is ignored.

What is claimed is:

1. A composition which is suitable for use as a reagent in assaying a sample, the composition comprising a plurality of particles, each of the particles
(i) having a coding characteristic,
(ii) containing analyte-interaction sites, and
(iii) belonging to one only of a plurality of defined categories, and each of the particles in each defined category
(a) having the same coding characteristic, the coding characteristic being provided by a single coding label in a preselected amount or by two or more coding labels in preselected amounts, the coding label or labels being selected from a group of at least n labels, where n is at least 3, all of the labels being capable of being assessed in the same way, the number of coding labels on each particle being at most (n−1), and each of the labels in the group being a coding label in at least one of the defined categories, and
(c) containing the same analyte-interaction sites; ps the combination of the coding characteristic and the analyte-interaction sites on the particles in each category being different from the combination of the coding characteristic and the analyte-interaction sites in all the other categories.

2. A composition according to claim 1 wherein n is 4.

3. A composition according to claim 2 wherein each of the particles belongs to a first class (AB) or to a second class (XY),
each of the particles of class AB belonging to one of two or more subclasses (AB₁, AB₂ . . . ABₙ),
each of the particles in each subclass AB₁, AB₂ . . . ABₙ being coded by at least one of a first label A and a second label B, the amounts of the labels A and B being such that the particles of each sub class can be distinguished from the particles of the other subclasses AB₁, AB₂ . . . ABₙ,
each of the particles in each subclass AB₁, AB₂ . . . ABₙ belonging to one of two subsubclasses (AB_{1X}, AB_{1Y}), (AB_{2X}, AB_{2Y}) . . . (AB_{nX}, AB_{nY}), and
each of the particles of class XY belonging to one of two or more subclasses (XY₁, XY₂ . . . XYₙ),
each of the particles in each subclass XY₁, XY₂ . . . XYₙ being coded by at least one of a third label X and a fourth label Y, the amounts of the labels X and Y being such that the particles in each sub class can be distinguished from the particles of the other subclasses XY₁, XY₂ . . . XYₙ;
each of the particles in each subclass XY₁, XY₂ . . . XYₙ belonging to one of two subsubclasses (XY_{1A}, XY_{1B}), (XY_{2A}, XY_{2B}) . . . (XY_{nA}, XY_{nB}),
the analyte-interaction sites contained by the particles in each of the subsubclasses (AB_{1X}, AB_{1Y}), (AB_{2X}, AB_{2Y}) . . . (AB_{nX}, AB_{nY}) and (XY_{1A}, XY_{1B}), (XY_{2A}, XY_{2B}) . . . (XY_{nA}, XY_{nB}), being different from the analyte-interaction sites contained by the particles in the other subsubclasses.

4. A composition according to claim 3 wherein each of the labels is a fluorochrome.

5. A composition according to claim 3 wherein each of the particles is a single assay particle.

6. A composition according to claim 3 wherein some of the particles are dual assay particles and the remainder are single assay particles.

7. A composition which is a precursor for a composition according to claim 1 and which is the same as said composition except that at least some of the particles contain, in place of the analyte-interaction sites, precursors for the analyte-interaction sites.

8. A composition which can be examined in a cytometer or like instrument, the composition comprising a plurality of particles, each of the particles
(i) having a coding characteristic,
(ii) containing analyte-interaction sites and/or corresponding sites which are the same as said analyte-interaction sites except that they have interacted with one or more analytes, and
(iii) belonging to one only of a plurality of defined categories, each of the particles in each defined category
(a) having the same coding characteristic, the coding characteristic being provided by a single coding label in a preselected amount or by two or more coding labels in preselected amounts, the coding label or labels being selected from a group of at least n labels, where n is at least 3, all of the labels being capable of being assessed in the same way, the number of coding labels on each particle being at most (n−1), and each of the labels in the group being a coding label in at least one of the defined categories, and
(b) containing the same analyte-interaction sites and/or corresponding sites which have interacted with one or more analytes;
the combination of the coding characteristic and the analyte-interaction sites and/or corresponding sites on the particles in each category being different from combination of the coding characteristic and the analyte-interaction sites and/or corresponding sites on the particles in all the other categories; and
the particles in at least one of the categories containing analyte-interaction sites which have interacted with one or more analytes and which are associated with a signal label which (i) is selected from the group of at least n labels and (ii) is not one of the labels which provides the coding characteristic for the particle containing the interacted site.

9. A composition according to claim 8 wherein n is 4.

10. A composition according to claim 9 which is the same as a composition as defined in claim 3 except that analyte-interaction sites on the particles in at least one of the subsubclasses have interacted with one or more analytes, and those interacted sites are associated with a signal label which is
(i) for the analyte-interaction sites on the particles of class AB, the third label X for one of the subsubclasses of each subclass and the fourth label Y for the other of the subsubclasses of that subclass, and
(ii) for the analyte-interaction sites on the particles of class XY, the first label A for one of the subsubclasses of each subclass and the second label B for the other of the subsubclasses of each subclass;

11. A composition according to claim 10 wherein each of the labels is a fluorochrome.

12. A composition according to claim 10 wherein each of the particles is a single assay particle.

13. A composition according to claim 10 wherein some of the particles are dual assay particles and the remainder are single assay particles.

14. A method of analyzing a sample to ascertain whether it contains possible analytes, the method comprising
(A) contacting the sample with a composition according to claim 1;
(B) before, during or after step (A), carrying out a treatment which results in the association of a signal label with the sites which have interacted with one or more analytes, the signal label (i) being selected from the group of at least n labels and (ii) not being one of the labels which provides the coding characteristic for the particles carrying the interacted sites; and
(C) after steps (A) and (B), identifying the coding and signal labels on each particle of a representative sample of the particles.

15. A method according to claim 14 wherein step (B) comprises
(B1) before, during or after step (A), carrying out a treatment which, for each of the possible analytes and group or groups of analytes, if present, results in the presence of a signal label as defined on analyte-interaction sites which have interacted with the analyte or one of the group or groups of analytes.

16. A method according to claim 14 wherein step (B) comprises
(B2) before, during or after step (A), contacting the sample, or the reagent, or the product of step (A), with a composition comprising an analog for each of the possible analytes or group of possible analytes, whereby analyte-interaction sites which have not interacted with the analytes interact with said analogs, and before, during or after said contacting, carrying out a treatment which, for each analog, results in the presence of a signal label as defined on the analyte-interaction sites which have interacted with one of the analogs.

17. A method according to claim 14 wherein n is 4, and each of the particles belongs to a first class (AB) or to a second class (XY),
each of the particles of class AB belonging to one of two or more subclasses (AB₁, AB₂ . . . ABₙ),
each of the particles in each subclass AB₁, AB₂ . . . ABₙ being coded by at least one of a first label A and a second label B, the amounts of the labels A and B being such that the particles of each sub class can be distinguished from the particles of the other subclasses AB₁, AB₂ . . . ABₙ,
each of the particles in each subclass AB₁, AB₂ . . . ABₙ belonging to one of two subsubclasses (AB_{1X}, AB_{1Y}), (AB_{2X}, AB_{2Y}) . . . (AB_{nX}, AB_{nY}), and
each of the particles of class XY belonging to one of two or more subclasses (XY₁, XY₂ . . . XYₙ),
each of the particles in each subclass XY₁, XY₂ . . . XYₙ being coded by at least one of a third label X and a fourth label Y, the amounts of the labels X and Y being such that the particles in each sub class can be distinguished from the particles of the other subclasses XY₁, XY₂ . . . XYₙ;
each of the particles in each subclass XY₁, XY₂ . . . XYₙ belonging to one of two subsubclasses (XY_{1A}, XY_{1B}), (XY_{2A}, XY_{2B}) . . . (XY_{nA}, XY_{nB}),
the analyte-interaction sites contained by the particles in each of the subsubclasses (AB_{1X}, AB_{1Y}), (AB_{2X}, AB_{2Y}) . . . (AB_{nX}, AB_{nY}) and (XY_{1A}, XY_{1B}), (XY_{2A}, XY_{2B}) . . . (XY_{nA}, XY_{nB}), being different from the analyte-interaction sites contained by the particles in the other subsubclasses.

18. A method according to claim 17 wherein each of the labels is a fluorochrome, and some of the particles are dual assay particles and the remainder are single assay particles.

19. A cytometer comprising a computer which is programmed so that the cytometer will examine a composition according to claim 8 and produce an assay of the analytes which have interacted with the analyte-interaction sites.

20. Software which can be installed on a computer controlling a cytometer so that the cytometer will examine a composition according to claim 8 and produce an assay of the analytes which have interacted with the analyte-interaction sites.
